(19) United States
(12) Patent Application Publication
(10) Pub. No.: US 2011/0091862 A1
(43) Pub. Date: Apr. 21, 2011

(54) GENERATION OF AN HLA-NEGATIVE OSTEOGENIC PRECURSOR CELL LINE

(75) Inventors: Mark S.F. Clarke, Houston, TX (US); Mark Brinker, Houston, TX (US); Alamelu Sundaresan, Sugarland, TX (US)

(73) Assignee: OSTEOSPHERE, LLC, Houston, TX (US)

(21) Appl. No.: 12/742,831

(22) PCT Filed: Nov. 13, 2008
(86) PCT No.: PCT/US08/83443

§ 371 Date: Dec. 15, 2010

Related U.S. Application Data
(60) Provisional application No. 60/988,025, filed on Nov. 14, 2007.

(51) Int. Cl. C12N 5/077

(52) U.S. Cl. 435/1.1; 435/366

(57) ABSTRACT

The present disclosure provides methods for producing human osteogenic cell lines in which HLA Class I expression is absent or reduced. The resulting osteogenic cells can then be used, for example, to form mineralized three-dimensional bone constructs which can be used, for example, in bone grafting procedures. The lack of HLA Class I expression in the cells of the mineralized three-dimensional bone constructs reduces or eliminates the potential for host-graft rejection.

### CLAIM OF PRIORITY

The present application for patent claims priority to U.S. Provisional Application No. 60/988,025 entitled “Generation of an HLA-Negative Osteogenic Precursor Cell Line” filed Nov. 14, 2007, assigned to the assignee hereof, and hereby expressly incorporated by reference herein.

### FIELD OF THE INVENTION

The invention relates to the preparation of human osteogenic cell lines which may be used to prepare ex vivo-derived mineralized three-dimensional bone constructs which replicate natural bone.

### BACKGROUND

One of the central problems associated with studying both the normal and pathophysiology of bone is that as an organ system it is slow growing and the time to show an observable response to a particular stimulus is relatively long. The nature of the mineralized tissue matrix of bone in vivo and its complex architecture also presents several technical problems associated with how experimental observations can be made. At present, truly informative studies designed to understand bone physiology have relied primarily on the removal of samples of bone tissue from normal or diseased tissue either in a clinical setting or from experimental animal models.

To date, there is no three dimensional tissue culture model of bone, either of animal or human origin. The prior art has relied primarily on the use of monotype cell type cultures of osteoblasts or osteoclast cells grown on planar, two dimensional tissue culture surfaces. Such cultures have also been grown in three dimensional collagen support gels and some investigators have utilized culture systems that allow types of mechanical strain to be applied to the cells in order to study the effects of mechanical loading. However, these cultures have been primarily focused on the responses of a single cell type, such as osteoblasts, to various environmental stimuli.

Existing planar monotype tissue culture models of bone do not allow the study of the interactions between the different cell types present in normal bone responsible for normal bone remodeling. The developmentally inactive osteocyte cell type present in the mineralized matrix of normal bone in vivo (from which osteoblasts are derived) have yet to be fully characterized in any tissue culture model due to their supposed transformation into osteoblasts once they have been removed from the bone matrix and placed into culture.

Moreover, the process of mineralization, which is essential to the formation of new bone, has previously only been studied in monotype cultures of osteoblasts. The mineralization process has been studied in such models in the absence of the major cell type involved in the removal of mineralized material, namely the osteoclast. However, the complex interplay between both of these cell types is essential for normal bone remodeling (i.e. bone formation and bone loss). Without both cell types being present, a true in vitro/ex vivo representation of the normal or indeed pathological processes involved in the bone remodeling process is impossible. As such, the use of such monotype culture models to investigate the effects of manipulations, such as anti-osteoporotic drugs or mechanical load interventions, have limited utility due to the lack of similarity to the true physiological state existing within bone tissue in vivo.

### SUMMARY

In one aspect, the disclosure provide methods for producing a human osteogenic cell line in which HLA Class I expression is silenced. In one embodiment, the method comprises providing adult mesenchymal stem cells or embryonic mesenchymal stem cells; introducing a short hairpin RNA (shRNA) cassette capable of directing expression of an inhibitory RNA specific for the HLA Class I gene into the adult mesenchymal stem cells or the embryonic mesenchymal stem cells; and identifying and propagating individual cells in which HLA Class I RNA is silenced. The resulting osteogenic cells can then be used, for example, to form mineralized three-dimensional bone constructs which can be used, for example, in bone grafting procedures. The lack of HLA Class I expression in the cells of the mineralized three-dimensional bone constructs reduces or eliminates the potential for host-graft rejection.

### BRIEF DESCRIPTION OF THE DRAWINGS

FIG. 1 is a flowchart of an example of a method for preparing mineralized three-dimensional bone constructs.

FIGS. 2A-2B present images of mineralized three-dimensional bone constructs at 14 days of mineralization (FIG. 2A) and 21 days of mineralization (FIG. 2B). The scale bars each represent 1 cm.

FIGS. 3A-3B present images of mineralized three-dimensional bone constructs. FIG. 3A presents a fluorescence confocal microscopy image of an optical section through bone constructs in which the osteoclast precursor cells were labeled with a fluorescent cell tracking dye (observable as white spots in FIG. 3A). FIG. 3B shows the same constructs viewed in incident laser light (i.e. non-fluorescent illumination) to illustrate the shape of the constructs. The scale bar in each of FIG. 3A and FIG. 3B is 200 μm.

FIG. 4 presents a three dimensional reconstruction of a large bone construct using Z series confocal imaging. Osteoclast precursors were labeled with a fluorescent cell tracking dye. Panels A-I in FIG. 4 are the individual images used by the confocal imaging software to build the optical reconstruction of the bone construct in three dimensions, each image representing a sequential view over the surface of the construct (white spots indicate individual labeled osteoclast cells). Panel J is a single image of the surface of a large bone construct in which structures reminiscent of resorption pits or lacunae found in actively remodeling bone in vivo can be clearly seen formed by labeled osteoclasts on the surface of the OsteoSphere (indicated by arrows, Bar equals 300 microns).

FIGS. 5A-5D show Alizarin red S staining and von Kossa staining of sections through a bone construct. FIG. 5A shows a 5× magnification image of Alizarin red S staining and FIG. 5B shows a 20× magnification image of Alizarin red S staining (which appears as the dark regions of the images). FIG. 5C shows a 5× magnification image of von Kossa staining, and FIG. 5D shows a 20× magnification image of von Kossa staining (which appears as the dark regions of the images). FIG. 5E shows a composite low power image of a complete 10 micron thick frozen cross-section of a Bouin's fixed OsteoSphere stained with Alizarin red S.

FIGS. 6A-6B show Harris Hematoxylin staining of sections through a bone construct. FIG. 6A is a 5× magnification image and FIG. 6B is a 20× magnification image. The dark regions of the image indicate staining Arrows in FIG. 6B point to large numbers of cells embedded within the crystalline matrix in the three dimensional construct.

FIGS. 7A-7C show images of bone construct in which osteoclast precursors were labeled with a fluorescent cell tracking dye prior to formation of the bone construct, and the bone construct was stained with a primary antibody against osteocalcein (a marker of osteoblast differentiation) and an Alexa 488-labeled secondary antibody. FIG. 7A shows osteocalcein staining, FIG. 7B shows CellTracker-Orange staining, and FIG. 7C shows the same construct illuminated with incident laser light. The results indicate that osteocalcein staining and cell tracking dye (both visible as a white “ring” around the construct in FIGS. 7A and 7B) are spatially localized to the same area of the construct.

FIG. 8 shows the results of a real-time quantitative PCR assay analysis of mRNA extracted from mineralized bone construct material.

FIG. 9 depicts an impact-mediated loading apparatus known as the G-loader.

FIG. 10 shows fluorescent micrographs demonstrating enhancement of impact-mediated loading of IgG (fluorescently labeled) into Swiss 3T3 cells under hypergravity conditions. Panel A shows control cells (no impact-mediated loading), Panel B shows impact-mediated loading at 1×g, and Panel C shows impact-mediated loading at 200×g.

FIG. 11 shows fluorescent micrographs of cells that experienced impact-mediated loading at 200×g of a plasmid construct encoding for a green fluorescent protein. The cells used were primary human skeletal myoblasts (HSKMC), Swiss 3T3 cells (3T3) and primary bovine capillary endothelial cells (BCEC).

### DETAILED DESCRIPTION

In one aspect, the present disclosure provides mineralized three-dimensional bone constructs (sometimes referred to as “OsteoSpheres” or simply as “bone constructs”). The mineralized three dimensional constructs of the disclosure are “bone like” in appearance by visual inspection, in certain important respects resembling trabecular bone (also known in the art as “spongy bone”). In preferred embodiments, the mineralized three-dimensional bone constructs of the disclosure are macroscopic in size and are approximately spheroidal in shape, preferably between about 200 μm and about 4 mm in diameter; however, larger and smaller bone constructs are specifically contemplated.

The bone constructs comprise an inner core surrounded by an outer layer. The inner core comprises a three-dimensional crystalline matrix that stains positively with Alizarin Red S stain and with the von Kossa histochemical stain, indicating that it comprises mineral elements observed in normal human bone in vivo, including calcium, phosphates, and carbonates. The inner core also comprises osteoblasts and/or osteocytes embedded within the crystalline matrix, and is preferably devoid of necrotic tissue. Osteocytes are developmentally inactive cells found only in native bone tissue in vivo and are believed to be formed from osteoblasts that have become trapped in the crystalline matrix. The outer layer is comprised of osteoclasts. The cell types in the bone constructs of the disclosure can be obtained from any mammalian species, but are preferably obtained from humans.

In another aspect, the disclosure provides methods for producing the mineralized three-dimensional bone constructs. In general, the bone constructs of the disclosure are produced by culturing osteoclast precursors and osteoblasts together under randomized gravity vector conditions (approaching those conditions that cultured cells experience during microgravity culture) in a matrix-free culture medium. Osteoclast precursors may be obtained from bone marrow and/or peripheral blood lymphocytes by techniques well known in the art. Osteoclast precursors may also be obtained from commercial sources (for example, from Cambrex/Lonza, Inc.). Osteoblasts, preferably primary human osteoblasts, may also be obtained by techniques well known in the art, and may also be obtained from commercial sources (for example, from PromoCell, Inc. and from Cambrex/Lonza, Inc.). A “matrix-free culture medium” is a cell culture medium which does not include carrier material (such as microcarrier beads or collagen gels) onto which osteoblasts and osteoclast precursors can attach. Suitable cell culture media include Eagle's Minimal Essential Medium (EMEM) or Dulbecco's Modified Eagle's Medium (DMEM), preferably supplemented with fetal bovine serum (FBS). Preferably, the matrix-free culture medium also comprises osteoblast growth supplements such as ascorbic acid. The matrix-free culture medium preferably also further comprises osteoclast differentiation factors, such as Receptor Activator of NF-kB (RANK) ligand and macrophage colony stimulating factor (M-CSF). For example, in one embodiment the matrix-free culture medium comprises FBS-supplemented DMEM, ascorbic acid, RANK ligand, and M-CSF. Example 2 includes a description of one suitable matrix-free culture medium.

The osteoclast precursors and the osteoblasts are cultured together under randomized gravity vector conditions effective to achieve the formation of mixed aggregates of the two cell types. The aggregates are then further cultured under randomized gravity vector conditions to increase the aggregates size and to differentiate the osteoclast precursors into mature osteoclasts.

After a predetermined time, the aggregates are cultured under randomized gravity vector conditions in a matrix-free mineralization culture medium. A “matrix-free mineralization culture medium” is a cell culture medium that includes one or more mineralization agents, such as osteoblast differentiation factors, that induce osteoblasts to produce crystalline deposits (comprising calcium, phosphate, and carbonates) but which does not include carrier material (such as microcarrier beads and collagen gels) onto which osteoblasts and osteoclast precursors can attach. For example, in one embodiment, a matrix-free mineralization culture medium comprises FBS-supplemented EMEM or DMEM, supplemented with the osteoblast differentiation factors. Osteoblast differentiation factors include beta-glycerophosphate and hydrocortisone-21-hemisuccinate. Preferably, the matrix-free mineralization culture medium also includes osteoclast differentiation factors such as RANK ligand and M-CSF, and also includes osteoblast growth supplements such as ascorbic acid. For example, in one embodiment the matrix-free mineralization culture medium comprises FBS-supplemented DMEM, beta-glycerophosphate, ascorbic acid, hydrocortisone-21-hemisuccinate, RANK ligand and M-CSF. Example 2 includes a description of one suitable matrix-free mineralization medium.

In preferred embodiments, randomized gravity vector conditions are obtained by culturing osteoclast precursors and osteoblasts in a low shear stress rotating bioreactor. Such bioreactors were initially designed to mimic some of the physical conditions experienced by cells cultured in true microgravity during space flight. In general, a low shear stress rotating bioreactor comprises a cylindrical culture vessel. One or more ports are operatively associated with the lumen of the vessel for the introduction and removal of cells and culture media. The cylindrical culture vessel is completely filled with a culture medium to eliminate head space. The cylindrical culture vessel rotates about a substantially central horizontal axis. The resulting substantially horizontal rotation occurs at a rate chosen so that (1) there is essentially no relative motion between the walls of the vessel and the culture medium; and (2) cells remain in suspension within a determined spatial region of the vessel such that they experience a continuous “free fall” through the culture medium at terminal velocity with low shear stress and low turbulence. This free fall state may be maintained continuously for up to several months in some applications described in the prior art. The continuous orbital movement of the medium relative to the cells also allows for highly efficient transfer of gases and nutrients.

In some embodiments, the diameter of the cylindrical culture vessel is substantially greater than its height. Such cylindrical culture vessels are often referred to in the art as High Aspect Ratio Vessels (HARVs). For example, a HARV having a volume of 10 mL may have a diameter of about 10 cm and a height of about 1 cm. At least a portion of the vessel walls may be comprised of a gas permeable membrane to allow gas exchange between the culture medium and the surrounding incubator environment. A suitable HARV is described in, for example, U.S. Pat. No. 5,437,998, incorporated by reference herein in its entirety. One commercial embodiment of a HARV is the Rotating Cell Culture System (RCCS) available from Synthecon, Inc.

In some embodiments, the diameter of the cylindrical culture vessel is substantially smaller than its height. Such cylindrical culture vessels are often referred to in the art as Slow Turning Lateral Vessels (STLVs). STLVs typically have a core, comprised of a gas permeable membrane, running through the center of the cylinder in order to allow gas exchange between the culture medium and the surrounding incubator environment. STLVs are available from Synthecon, Inc.

The use of low shear stressing rotating bioreactor culture systems is described in, for example, Nickerson et al., Immunity. 69:7106-7120 (2001); Carterson et al., Infection & Immunity. 73(2):1129-40 (2005); and in Goodwin et al. U.S. Pat. No. 5,496,722, each of which is specifically incorporated herein by reference in its entirety.

In one embodiment, osteoclast precursors and osteoblasts are introduced into a cylindrical culture vessel in matrix-free culture medium. The osteoclast precursors and the osteoblasts may be introduced into the cylindrical culture vessel separately, or they may be introduced into the cylindrical culture vessel as a pre-mixture of the two cell types. Preferably, the cells are introduced into the cylindrical culture vessel at a osteoblast:osteoclast precursor ratio of from about 2:1 to about 3:1, although higher and lower ratios are within the scope of the disclosure. The absolute number of cells introduced into the cylindrical culture vessel may also be varied. For example, in some embodiments where a ratio of about 2:1 is employed, about 2 million osteoblasts and about 1 million osteoclast precursors are introduced; in other embodiments about 4 million osteoblasts and about 2 million osteoclast precursors are introduced; and in still further embodiments about 8 million osteoblasts and about 4 million osteoclast precursors are introduced. The ratio of osteoblasts:osteoclast precursors and the absolute number of cells can be varied in order to vary the size and the number of aggregates formed. In addition, other cell types may also be introduced into the cylindrical culture vessel. For example, bone marrow stroma and stem cells may be cultured along with the osteoblasts and the osteoclast precursors.

One or more cell types may optionally be labeled with a cell-tracking marker, such as a fluorescent cell-tracking dye, prior to their introduction into the cylindrical culture vessel. In this way, it is possible to determine the location of the individual cell types during, or at the conclusion of, the formation of the bone constructs. For example, fluorescent CellTracker dyes, available from Invitrogen, Inc., may be used in conjunction with fluorescence microscopy techniques, such as confocal fluorescence microscopy. If more than one cell type is labeled, then they are labeled with different colored dyes so that each cell type can be tracked independently.

Cells are then cultured in the matrix-free culture medium in the cylindrical culture vessel during substantially horizontal rotation to form aggregates of the two cell types. The rate of substantially horizontal rotation during the aggregation phase is chosen so that both (1) low shear conditions are obtained; and (2) the osteoclast precursors and the osteoblasts are able to coalesce and form aggregates. The rate of substantially horizontal rotation may be selected by monitoring the cylindrical culture vessel and by monitoring the cells and aggregates in the cylindrical culture vessel (for example using microscopy), to insure that the cells and aggregates are not sedimenting (which may be caused by too low a rate of rotation) or experiencing mechanical or excessive hydrodynamic shear stress. In embodiments in which a HARV is used, osteoclast precursors and osteoblasts may form a “boundary” layer situated in the middle of the HARV during the aggregation phase.

Preferably, the rate of substantially horizontal rotation during the aggregation phase is lower than the rate typically used for culturing cells. For example, in embodiments where the cylindrical culture vessel is a 10 mL HARV having a diameter of about 10 cm and a height of about 1 cm, substantially horizontal rotation at less than about 14 revolutions per minute (rpm) may be used. More preferably, substantially horizontal rotation at less than about 12 rpm is used. In certain preferred embodiments, substantially horizontal rotation at between about 1 rpm and about 4 rpm is used. In one specific embodiment, substantially horizontal rotation at about 2 rpm is used. Note that the aforementioned rpm values are provided with reference to a 10 mL HARV having the aforementioned dimensions. The rpm values will vary depending on the volume and dimensions of the cylindrical culture vessel. The rpm values during the aggregation phase for all such vessels are easily determined using the aforementioned methodology.

Without being bound by a particular theory or mechanism, it is believed that the use of a matrix-free culture medium allows the use of rates of rotation that are substantially lower than previously reported in the art for culturing mammalian cells in a low shear stress rotating bioreactor. The use of low rotation rates, in turn, is believed for the first time to promote efficient association of osteoclast precursors and osteoblasts into aggregates, and to promote three-dimensional organization of these two cell types within the aggregates. Thus, the organization of the cell types within the aggregate is not constrained or influenced by an exogenous carrier material, but rather by native cell-cell interaction. Consequently, the three-dimensional organization of the osteoblasts and osteoclasts is physiologically realistic.

The rate of substantially horizontal rotation may optionally be adjusted periodically during the aggregation phase in order to compensate for the increase in the sedimentation velocity (which is a function of volume and density) of the forming aggregates, thereby maintaining the aggregates in low shear “free fall” and preventing impact with the vessel wall.

The aggregation phase proceeds for a period of time sufficient to produce the desired size of aggregates. Aggregate formation may be monitored during the aggregation phase by visual inspection, including through the use of microscopy. It will be apparent from the disclosure that the size of the aggregates is also dependent on the number of cells that are initially introduced into the cylindrical culture vessel, the length of time allowed for aggregation, as well as the rotation rate. In one example, the aggregation phase is allowed to proceed for between about 24 hours and about 48 hours.

Once aggregates of the desired size have formed, the aggregates are preferably further cultured in the cylindrical culture vessel during substantially horizontal rotation for a period of time sufficient to allow the aggregates to grow to a desired size through cell proliferation and/or to allow the osteoclast precursors in the aggregates to differentiate into osteoclasts. For example, the further culturing of the aggregates may proceed for between about 5 and about 7 days and may lead to grown aggregates having a diameter from between about 200 μm and about 4 mm. The resultant aggregates are sometimes referred to herein as “spheroids.” Preferably, the rate of substantially horizontal rotation during the further culturing is higher than the rate during the aggregation phase, but still provides low shear conditions in the cylindrical culture vessel. For example, a rotation rate of between about 9 rpm and about 16 rpm, preferably about 14 rpm, may be used during further culturing for the 10 mL HARV exemplified above. The rate of substantially horizontal rotation may optionally be adjusted periodically during the further culturing phase in order to compensate for the increase in the sedimentation pathway of the aggregates as they grow in size (and hence undergo changes in volume and density), thereby maintaining the growing aggregates in low shear “free fall” and preventing impact with the vessel wall.

Once aggregates have attained a desired size, a matrix-free mineralization culture medium is introduced into the cylindrical culture vessel and the aggregates are cultured during substantially horizontal rotation until they become mineralized (either partially mineralized or fully mineralized), thereby forming the mineralized three-dimensional bone constructs of the disclosure. For example, the mineralization process may proceed for between about 7 days and about 21 days depending on the size of the aggregates and the degree of mineralization required. Preferably, the rate of substantially horizontal rotation during such the mineralization process is higher than the rate during the aggregation phase, but still provides low shear conditions in the cylindrical culture vessel. For example, a rotation rate of between about 9 rpm and about 20 rpm, preferably about 14 rpm, may be used during the mineralization phase for the 10 mL HARV exemplified above. The rate of substantially horizontal rotation may optionally be adjusted periodically during the mineralization phase in order to compensate for the increase in the sedimentation pathway of the aggregates as they increase in mass, thereby maintaining the mineralizing aggregates in low shear “free fall” and preventing impact with the vessel walls.

Mineralized three-dimensional bone constructs are harvested once they have achieved the desired size and mass. In cylindrical culture vessels with one or more access ports, the bone constructs are removed through a part. When the bone constructs exceed the diameter of the port, the vessel is disassembled to remove the bone constructs.

Osteoclasts and osteoblasts act coordinately in the mineralization process that occurs in vivo during bone formation and bone restructuring. Accordingly, the mineralized three-dimensional bone constructs of the disclosure, formed by the coordinated activity of osteoblasts and osteoclasts, are physiologically realistic.

As described above, the mineralized three-dimensional bone constructs of the disclosure mimic trabecular bone in many important aspects. The bone constructs of the disclosure therefore have a great many uses in the fields of, for example, physiology research and development, pharmaceutical research, and orthopedics. Without limitation, these include the direct benefit of developing a model for studying both normal bone physiology and the pathological responses observed in disease states such as osteoporosis, as well as providing a highly economical platform for drug development as it relates to the treatment of bone diseases.

The bone constructs of the disclosure also can be used for autologous grafts. Specifically, diseased or missing bone may be replaced with ex-vivo-derived mineralized three-dimensional bone constructs in which the component osteoclasts and osteoblasts are harvested from healthy bone and peripheral blood lymphocytes of the patient requiring the bone graft. Examples of pathologies where the bone constructs of the disclosure have therapeutic utility include fractures, non-unions of fractures, congenital deformities of bone, bone infections, bone loss, segmental bone defects, bone tumors, metabolic and endocrine disorders affecting bone, and tooth loss.

The bone constructs of the disclosure can also be used for allogenic (allograft) grafts. Specifically, diseased or missing bone can be replaced with ex vivo-derived mineralized three-dimensional bone constructs in which the component osteoclasts and osteoblasts are harvested from healthy bone and peripheral blood lymphocytes of another donor for the benefit of a patient requiring bone graft. Examples of pathologies where the bone constructs of the disclosure have therapeutic utility include fractures, non-unions of fractures, congenital deformities of bone, bone infections, bone loss, segmental bone defects, bone tumors, metabolic and endocrine disorders affecting bone, and tooth loss.

Because the bone constructs of the disclosure closely resemble bone formed in vivo, it is expected that they produce unique factors and/or cytokines essential for bone remodeling. Accordingly, the bone constructs of the disclosure serve as a source for identification and harvesting of these factors.

The bone constructs of the disclosure may also be used to study the interface between prosthetic devices/materials and bone tissue.

Sensors or stimulation devices may be incorporated into the bone constructs of the disclosure, and the resulting constructs implanted into bone tissue in vivo.

The bone constructs of the disclosure also may be used in the production of large structures of specific dimensions for “form-fitted” applications such as replacement of large regions of the skeleton. This may be achieved using a combination of tissue scaffolding/synthetic support materials embedded with numerous bone constructs to generate a much larger composite tissue aggregate.

The bone constructs of the disclosure also provide a low cost alternative in which to study the effects of microgravity, and of other space environment insults, such as radiation, on the process of bone formation/bone loss.

The following examples are not to be construed as limiting the scope of the invention disclosed herein in any way.

### EXAMPLES

### Example 1

### Flow Chart of a Method for Producing Bone Constructs

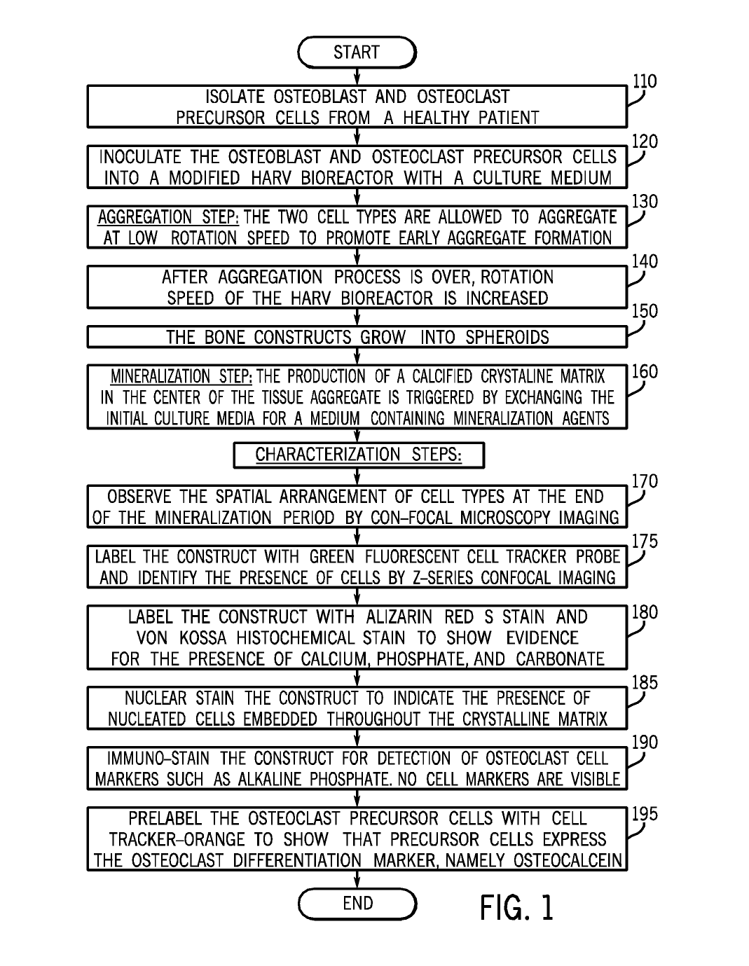

A flow chart of the method for producing mineralized three-dimensional bone constructs is provided in FIG. 1. Osteoblast and osteoclast precursor cells are first isolated (110) from a healthy patient and then inoculated (120) into a modified High Aspect Rotating Vessel (HARV) with a matrix-free culture medium. Cells are allowed to aggregate (130) at a rotation speed (typically 2 rpm) much lower than that commonly used for the culture of mammalian cells. Low speed promotes aggregation of the two or more cell types in the early stages of aggregate formation. After the aggregation period is over, the rotation speed of the High Aspect Rotating Vessel is increased (140). This allows the bone construct to grow into spheroids (150) in a state of “free fall”. The mineralization step (160) is then initiated by exchanging the initial matrix-free culture medium for a matrix-free mineralization culture medium, which initiates the production of a calcified crystalline matrix in the center of the tissue aggregate. The bone constructs are then characterized. The spatial arrangement of the different cell types is observed by confocal microscopy imaging (170). The cells are visualized with Z-series confocal imaging (175) by pre-labeling the initial cell constituents of the construct with green fluorescent cell tracker probe. The presence of calcium, phosphate and carbonate is revealed by using Alizarin red S stain and Kossa histochemical stain (180), while the presence of nucleated cells embedded in the crystalline matrix is revealed by nuclear staining (185). Immuno-staining of the construct (190) shows that cell markers such as alkaline phosphate are absent from the cells embedded in the crystalline matrix. Finally, prelabeling of the osteoclast precursor cells with Cell Tracker-Orange (195) shows that precursor cells allowed to aggregate and organize under these culture conditions co-localize with those cells expressing the osteoblast differentiation marker, namely osteocalcein, as a surface layer of the OsteoSphere.

### Example 2

### Production of Bone Constructs in a HARV

Cryopreserved primary normal human osteoblast cells and normal human osteoclast precursor cells were purchased from the Cambrex Corporation (East Rutherford, N.J.) and stored frozen under liquid nitrogen until needed.

Osteoblast cells were rapidly thawed by placing the vial in a 37° C. oven, removing the cell suspension from the vial and placing it in a 15 ml centrifugation tube and then diluting the cell suspension with 10 ml of Dulbecco's Modified Essential Medium (DMEM) supplemented with 10% (v/v) fetal bovine serum (10% FBS-DMEM). The cells were then collected by centrifugation at 100×g for 5 min at 4° C. The supernatant was then removed and the cell pellet was resuspended by gentle tituration in 10 ml of fresh 10% FBS-DMEM supplemented with 5 μM ascorbic acid and 1 mg/ml GA-1000 (gentamicin/amphotericin B mixture). This process was carried out to wash away the cryopreservatives in which the osteoblast cells had been frozen.

The resulting cell suspension was then inoculated into a T-75 tissue culture flask and incubated at 37° C. in a 5% CO₂ atmosphere tissue culture incubator for a total period of seven days, with the medium being exchanged every three days. After seven days the osteoblast culture was approaching confluence and the osteoblast cells were harvested by removing the cells from the surface of the flask using trypsin/EDTA digestion followed by collection of the cells by centrifugation as above. The cell pellet was then gently resuspended in 20 ml of fresh 10% FBS-DMEM supplemented with 5 μM ascorbic acid and 1 mg/ml GA-1000. The resulting cell suspension was then inoculated into two T-75 tissue culture flasks and again cultured for an additional seven days. This process of osteoblast cell expansion continued until the cells had reached passage 5 (i.e. five expansion/population doubling cycles).

When the osteoblast cells had reached Passage 5 in culture they were harvested using trypsin/EDTA digestion followed by collection of the cells by centrifugation as above. The cell pellet was then gently resuspended in 10 ml of fresh 10% FBS-DMEM supplemented with 5 μM ascorbic acid, 100 U/ml penicillin and 100 ug/ml streptomycin, penicillin/streptomycin being substituted for GA-1000 at this point due to the potential negative effects of gentamicin on the capability of osteoblast cells to produce mineralized extracellular matrix. The resulting osteoblast cell suspension was counted using a hemacytometer to ascertain the number of osteoblast cells/ml. An aliquot of cell suspension containing a total of six million osteoblast cells was removed and placed in a separate 15 ml centrifugation tube in preparation for the addition of osteoclast precursor cells.

Osteoclast precursor cells were rapidly thawed by placing the vial in a 37° C. oven, removing the cell suspension from the vial and placing it in a 15 ml centrifugation tube and then diluting the cell suspension with 10 ml of Dulbecco's Modified Essential Medium (DMEM) supplemented with 10% (v/v) fetal bovine serum (10% FBS-DMEM). The cells were then collected by centrifugation at 100×g for 5 min at 4° C. The supernatant was then removed and the cell pellet was resuspended by gentle tituration in 1 ml of fresh 10% FBS-DMEM supplemented with 5 μM ascorbic acid, 100 U/ml penicillin and 100 ug/ml streptomycin. This process was carried out to wash away the cryopreservatives in which the osteoclast cells had been frozen.

The resulting osteoclast precursor cell suspension was counted using a hemacytometer to ascertain the number of osteoclast precursor cells/ml. An aliquot of cell suspension containing a total of two million osteoclast cells was removed and added to the 15 ml centrifuge tube containing the six million osteoblast cells. The volume of medium in the centrifuge tube was then was adjusted to a total of 10 ml by the addition of fresh 10% FBS-DMEM supplemented with 5 μM ascorbic acid, 100 U/ml penicillin and 100 ug/ml streptomycin. Finally, the 10 ml of medium containing both osteoblast and osteoclast cells was supplemented with 50 ng/ml macrophage colony stimulating factor (M-CSF) and 50 ng/ml of receptor activator of NF-kB (RANK) ligand.

The resulting osteoblast/osteoclast cell suspension was then inoculated into a 10 ml rotating cell culture system (RCCS) flask (also know as a High Aspect Ratio Vessel—HARV) (Synthecon, Inc.) and horizontally rotated at 2 RPM for a period of 24 hr to allow coalescence of the osteoblast and osteoclast cells into a solid, three dimensional tissue construct. After a period of 24 hr, the rotation speed of the HARV was increased to 14 RPM in order ensure that the tissue construct was maintained in an optimal position within the HARV, namely not touching or hitting the sides of the rotating HARV rather in a state of “free-fall” within the medium contained within the rotating HARV. The cell medium within the HARV was exchanged with 10 ml of fresh 10% FBS-DMEM supplemented with 5 μM ascorbic acid, 100 U/ml penicillin, 100 ug/ml streptomycin, 50 ng/ml macrophage colony stimulating factor (M-CSF) and 50 ng/ml of receptor activator of NF-kB (RANK) ligand (a matrix-free culture medium) after every fourth day of culture.

After a period of seven days of culture in the HARV under the above conditions the medium was exchanged for 10 ml of fresh 10% FBS-DMEM supplemented with 5 μM ascorbic acid, 100 U/ml penicillin, 100 ug/ml streptomycin, 50 ng/ml macrophage colony stimulating factor (M-CSF), 50 ng/ml of receptor activator of NF-kB (RANK) ligand, 200 μM hydrocortisone-21-hemisuccinate and 10 mM beta-glycerophosphate (a matrix-free mineralization culture medium). The hydrocortisone-21-hemisuccinate and beta-glycerophosphate were added to the medium to induce mineralization of the tissue construct by the osteoblasts. The cell medium within the HARV was exchanged with 10 ml of fresh 10% FBS-DMEM supplemented with 5 μM ascorbic acid, 100 U/ml penicillin, 100 ug/ml streptomycin, 50 ng/ml macrophage colony stimulating factor (M-CSF), 50 ng/ml of receptor activator of NF-kB (RANK) ligand, 200 μM hydrocortisone-21-hemisuccinate and 10 mM beta-glycerophosphate every fourth day until the tissue construct was harvested.

### Example 3

### Imaging of Bone Constructs

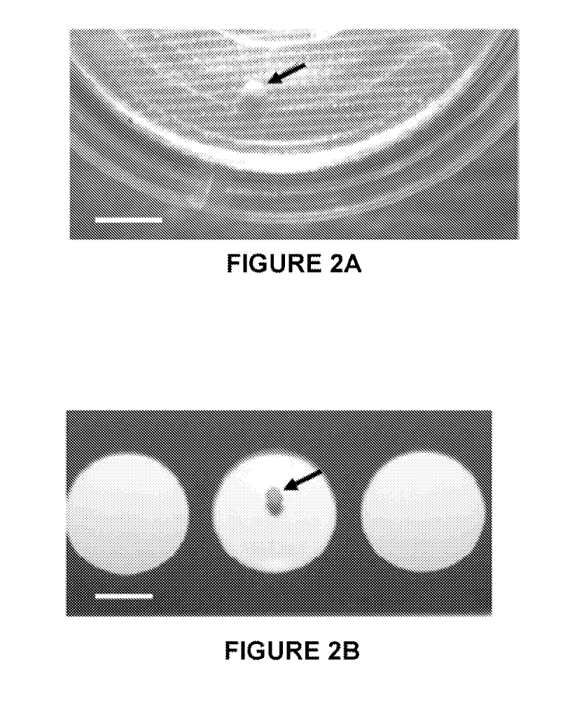

The method of Example 2 was followed, with the following differences: primary osteoblasts and osteoclast precursors were mixed together at about a 2:1 ratio of osteoblasts to osteoclast precursors, with the total number of cells being about 9 million cells; the mixture of cells was then horizontally rotated at 2 rpm for 48 hrs, and then at 14 rpm for 5 days; and mineralization proceeded at 16 rpm for 21 days. The resulting mineralized three-dimensional bone constructs are pictured in FIG. 2A (at 14 days of mineralization) and FIG. 2B (at 21 days of mineralization). The scale bar in each figure is 1 cm.

### Example 4

### Bone Constructs with Labeled Osteoclasts

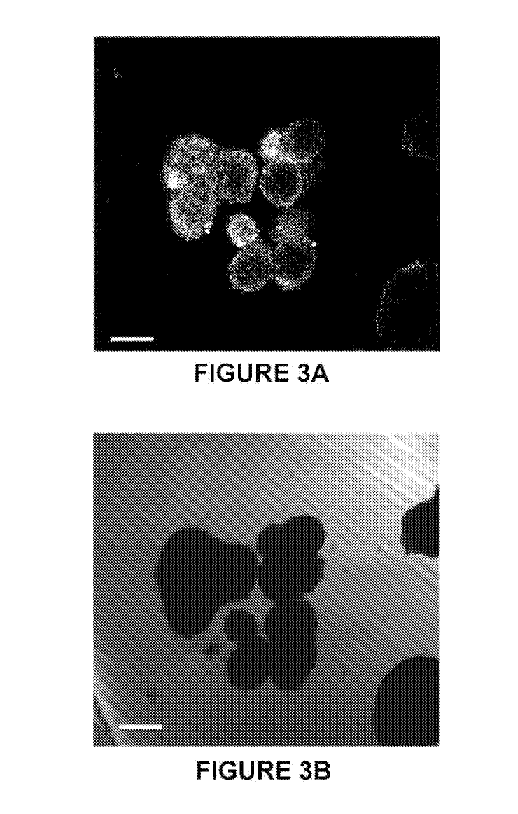

The method of Example 2 was followed, with the following differences: osteoclast precursors were labeled with the fluorescent CellTracker-Red probe (Invitrogen, Inc.) prior to mixing with osteoblasts; primary osteoblasts and labeled osteoclast precursors were mixed together at about a 2:1 ratio of osteoblasts to osteoclast precursors, with the total number of cells being about 3 million cells; the mixture of cells was then horizontally rotated at 2 rpm for 24 hrs, and then at 14 rpm for 5 days; and mineralization proceeded at 16 rpm for 14 days. FIG. 3A shows a fluorescence confocal microscopy image of an optical section through some of the resulting mineralized three-dimensional bone constructs. The results show that osteoclast precursor cells (observable as white spots in FIG. 3A) have spatially arranged themselves as an outer layer of the mineralized three-dimensional bone constructs with the putative osteoblast cells being embedded in the crystalline matrix of the central region of the constructs. FIG. 3B shows the same constructs viewed in incident laser light (i.e. non-fluorescent illumination) to illustrate the shape of the constructs. The scale bar in each of FIG. 3A and FIG. 3B is 200 μm.

### Example 5

### Optical Sectioning of a Bone Construct with Labeled Osteoclasts

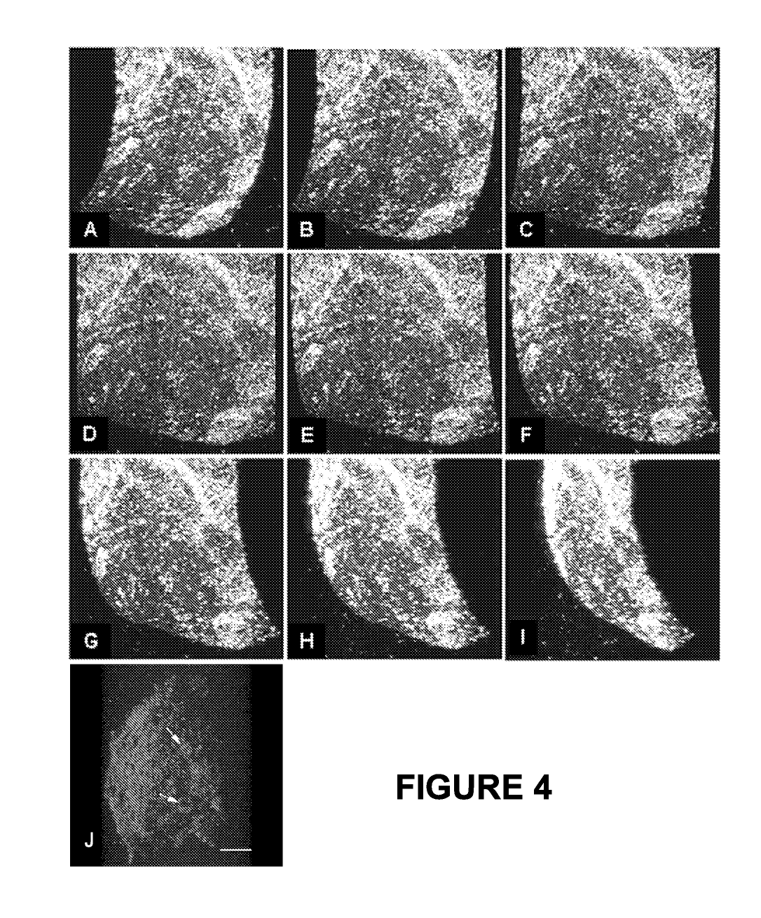

The method of Example 2 was followed, with the following differences: osteoclast precursors were labeled with the fluorescent CellTracker-Green probe (Invitrogen, Inc.) prior to mixing with osteoblasts; primary osteoblasts and labeled osteoclast precursors were mixed together at about a 2:1 ratio of osteoblasts to osteoclast precursors, with the total number of cells being about 6 million cells; the mixture of cells was then horizontally rotated at 2 rpm for 48 hrs, and then at 14 rpm for 5 days; and mineralization proceeded at 16 rpm for 21 days. Three dimensional reconstruction of a resulting large bone construct was performed using Z series confocal imaging. Panels A-I in FIG. 4 are the individual images used by the confocal imaging software to build the optical reconstruction of the bone construct in three dimensions, each image representing a sequential view over the surface of the construct (white spots indicate individual cells). FIG. 4 indicates that the arrangement of osteoclasts to the outer layer of the construct remains a feature of the construct even after extended culture periods (i.e. a total of four weeks in the HARV vessel, including three weeks grown in mineralization conditions). Labeled osteoclasts are apparent in an outer layer covering the surface of the construct. Additionally, structures reminiscent of resorption pits or lacunae found in actively remodeling bone in vivo can be clearly seen on the surface of the OsteoSphere in Panel J of FIG. 4 (indicated by arrows, Bar equals 300 microns).

### Example 6

### Staining of Bone Constructs with Alizarin Red S, von Kossa, and Harris Hematoxylin Stains

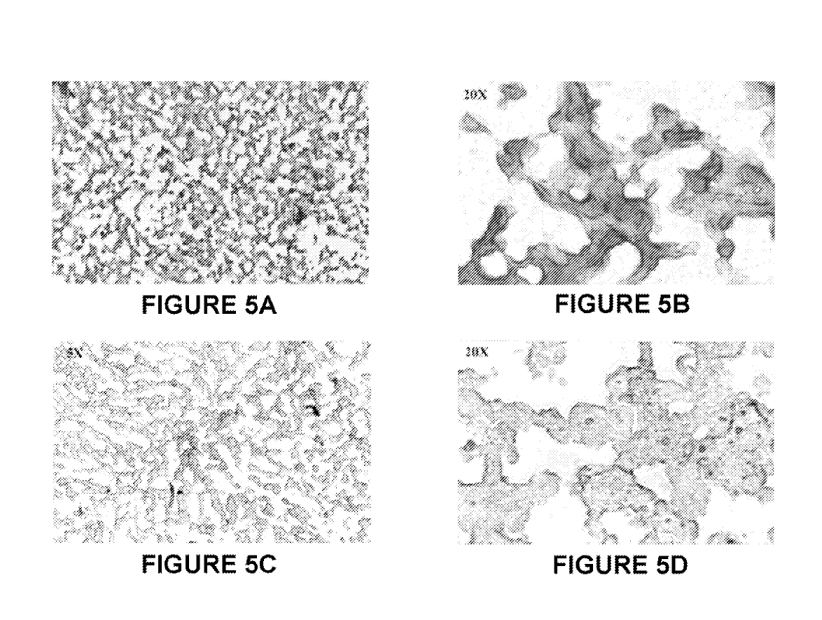

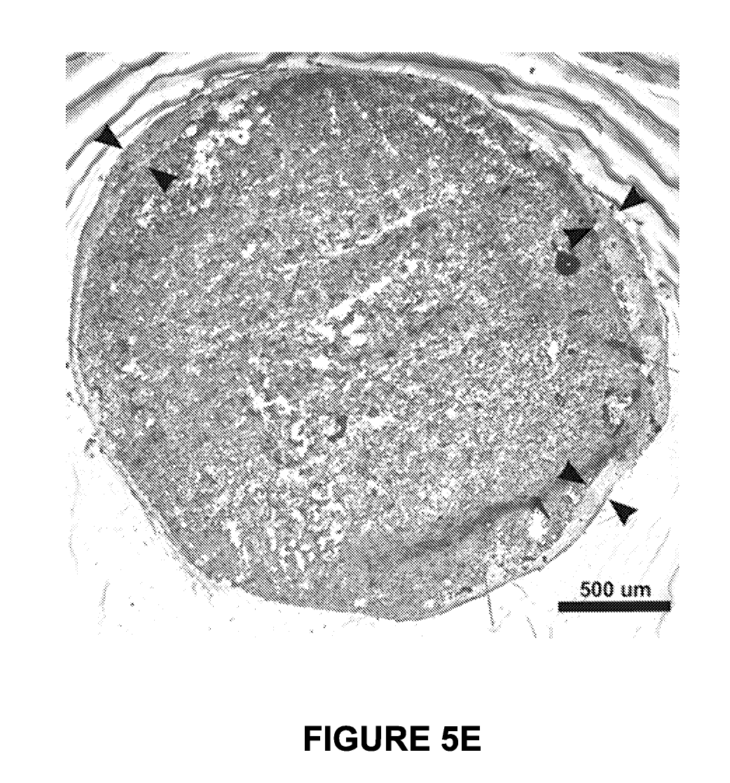

Mineralized three-dimensional bone constructs were prepared as detailed in Example 3. The bone constructs were then fixed using a Bouin's solution (a rapid penetrating fixative solution), frozen sectioned, and stained for calcium using the Alizarin red S stain and for phosphates and carbonates using the von Kossa histochemical stain. FIG. 5A shows a 5× magnification image of Alizarin red S staining and FIG. 5B shows a 20× magnification image of Alizarin red S staining (which appears as the dark regions of the images). FIG. 5C shows a 5× magnification image of von Kossa staining and FIG. 5D shows a 20× magnification image of von Kossa staining (which appears as the dark regions of the images). The results demonstrate that the crystalline matrix of the mineralized three-dimensional bone constructs contain mineral elements observed in normal human bone in vivo. In addition, when a composite, low-power image of a complete 10 micron thick frozen cross-section of a Bouin's fixed OsteoSphere stained with Alizarin red S was generated (FIG. 5E), an external zone (indicated by arrows) surrounding the OsteoSphere could be clearly discerned (Bar equals 500 microns). This outer zone surrounded the mineralized internal core of the OsteoSphere. This external zone of the OsteoSphere had been previously determined to contain osteoclast cells determined by confocal microscopy imaging as described in Examples 4 and 5 and shown in FIGS. 3 and 4.

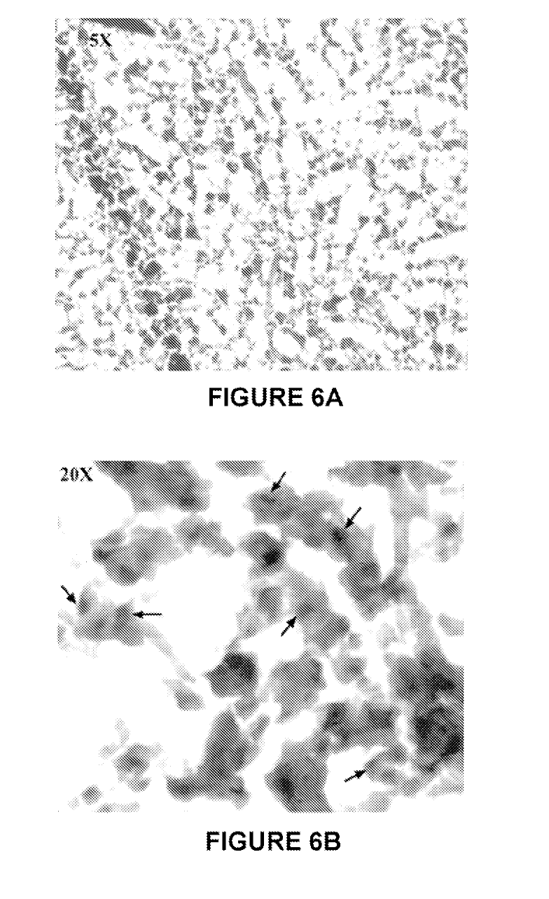

The same sections were also stained for the presence of nucleated cells using the Harris Hematoxylin stain. The results are shown in FIG. 6A (5× magnification image) and FIG. 6B (20× magnification image). The dark regions of the image indicate staining. The staining pattern illustrates a large number of cells embedded within the crystalline matrix of the three dimensional construct. These cell nuclei appear intact with little or no signs of nuclear fragmentation, a histological indicator of the occurrence of cell death/apoptosis. Arrows in FIG. 6B point to large numbers of cells embedded within the crystalline matrix in the three dimensional construct. Cell nuclei appear intact with little or no signs of nuclear fragmentation; such fragmentation would be a histological indicator of the occurrence of cell death/apoptosis. Immuno-staining of these sections for the presence of osteoblast cell markers, such as alkaline phosphatase, indicated the absence of osteoblast cell markers in the cell type embedded in the crystalline matrix. Thus, it is believed that the cells embedded in the crystalline matrix are osteocytes.

### Example 7

### Detection of Osteocalcein, an Osteoblast Differentiation Marker, in Bone Constructs Using Immunofluorescence

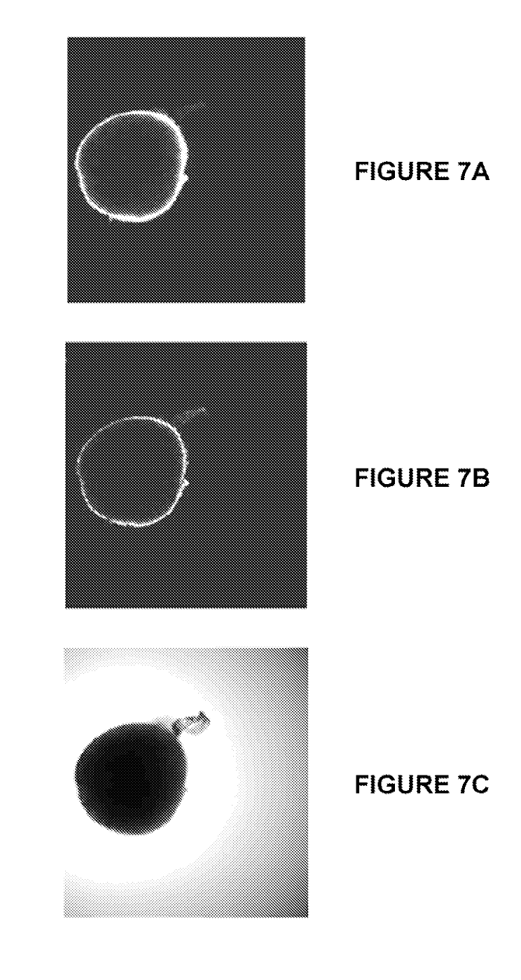

The method of Example 2 was followed, with the following differences: osteoclast precursors were labeled with the fluorescent CellTracker-Orange probe (Invitrogen, Inc.) prior to mixing with osteoblasts; primary osteoblasts and labeled osteoclast precursors were mixed together at about a 2:1 ratio of osteoblasts to osteoclast precursors, with the total number of cells being about 6 million cells; the mixture of cells was then horizontally rotated at 2 rpm for 48 hrs, and then at 14 rpm for 5 days; and mineralization proceeded at 16 rpm for 21 days. The resulting mineralized three-dimensional bone constructs were fixed using a phosphate buffered saline solution (pH 7.2) containing 1% (v/v) freshly generated formaldehyde. The fixed bone constructs were then immunochemically stained using a monoclonal antibody against osteocalcein (an osteoblast differentiation marker) as the primary antibody and an Alexa 488-labeled secondary antibody. FIG. 7A-7C shows images obtained by simultaneously imaging both markers in one of the bone constructs using confocal microscopy. Specifically, FIG. 7A shows osteocalcein staining, FIG. 7B shows CellTracker-Orange staining, and FIG. 7C shows the same construct illuminated with incident laser light. The results indicate that osteocalcein staining and CellTracker-Orange staining (both visible as a white “ring” around the construct in FIGS. 7A and 7B) are spatially localized to the same area of the construct. This indicates that the osteoclast precursor cells are localized to the same region as differentiated mature osteoblasts and that both were spatially localized to the surface of the construct.

### Example 8

### Demonstration of Bone Morphogenic Protein (BMP) Production by OsteoSpheres

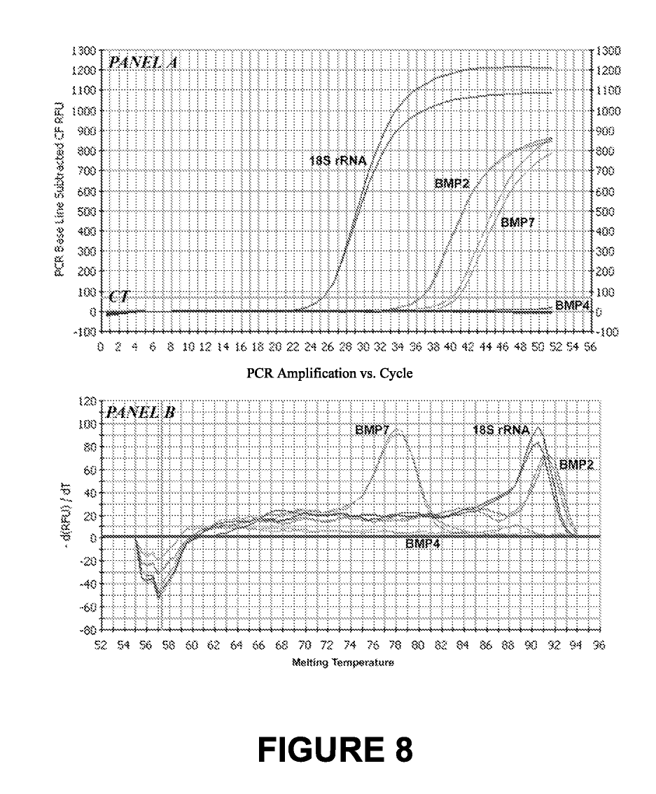

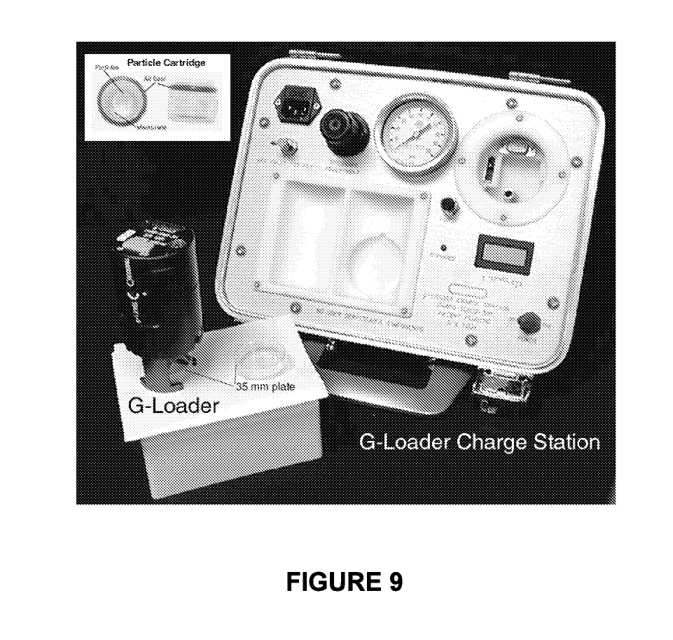

The method of Example 6 was followed for producing frozen sections of Bouin's fixed, mineralized OsteoSpheres grown for 21 days under mineralization conditions. A total of eight, 10 micron frozen sections of Bouin's fixed mineralized OsteoSpheres were collected and total RNA was extracted from the material using a micro-scale mRNA extraction/purification kit. The presence of intact mRNA in the extract was verified using a Pico™ Total mRNA Chip Assay (Agilent Technologies). The OsteoSphere-derived mRNA was then converted to cDNA and duplicate samples of cDNA where then probed with human sequence primer sets directed against sequences of either 18S ribosomal RNA (control), BMP-2, BMP-4 or BMP-7 using a real-time quantitative PCR assay (BioRad Laboratories). FIG. 8 demonstrates the expression of both BMP-2 and BMP-7 mRNA by OsteoSpheres as detected using a real-time quantitative PCR. Specifically, Panel A of FIG. 8 demonstrates that mature, 21 day old mineralized OsteoSpheres produced approximately eight times more BMP-2 mRNA than BMP-7 mRNA as indicated by CT values (“crossing the threshold”—horizontal line labeled CT, FIG. 8A) of 37 cycles for BMP-2 and approximately 40 cycles for BMP-7. In contrast, the CT value for the 18S ribosomal RNA control is approximately 25. No significant amount of BMP-4 mRNA was detected in the 21 day old mineralized OsteoSphere sample. Analysis of the melt curve generated for the assay indicates that the appropriate sized amplicons had been generated in the RT-qPCR assay (Panel B).

### Example 9

### Generation of an HLA-Negative Osteogenic Precursor Cell Line for Use in the Production of Three Dimensional Human Bone Constructs

The ideal bone graft material for use in long bone fractures/nonunions/defects and spinal fusion procedures is a material that has all of the characteristics of an autogenous bone graft without the need for a second surgical procedure to harvest the material. In addition, the material should preferably contain all those elements necessary to provide the optimal cellular environment for new bone formation and the promotion of fusion with the existing matrix of the bone being treated. By providing a bone graft material having these elements, bony fusion will occur in a predictable and efficient manner reducing the time required for the patient to heal.

Development of mineralized three-dimensional bone constructs (sometimes referred to as “OsteoSpheres”) using, for example, the methods disclosed in the preceeding examples, demonstrates that bone material similar to autogenous bone graft with both osteoinductive and osteoconductive properties can be generated using an ex vivo approach. As such, this approach can be used to produce autologous bone graft material using osteoblasts (harvested from normal healthy bone) and osteoclasts (harvested from peripheral blood lymphocytes) for a single patient. However, this autologous transplantation approach has limited utility for bone graft procedures in general. Although adult human bone is considered an immune privileged tissue under certain circumstances (i.e. exampled by the incorporation of cadaveric bone during allograft transplantation), transplantation of OsteoSpheres that contain living osteoblast, osteoclast and osteocyte cell types into a non-tissue type matched individual may induce a host-graft reaction.

Allograft bone material contains a variety of cellular material derived from dead cells contained in the mineral matrix (including a variety of growth factors and Bone Morphogenetic Proteins (BMPs)) but no living cells. The osteoconductive properties of allograft material center on the ability of the allograft to promote infiltration of the three dimensional mineralized matrix with osteogenic cells from the existing bone of the patient. These cells colonize the three dimensional matrix of the allograft and begin to incorporate the graft material into the existing bone matrix. During this process osteoclasts and osteoblasts infiltrate the allograft material; osteoclasts break down the mineralized allograft material and osteoblasts form new mineralized matrix until the allograft material has been fully integrated into the existing bone. This process can take several years to complete and the rate and efficiency of the incorporation is dependent on a variety of factors including the amount of BMP's found in the allograft and the availability of osteogenic cells from the patient's existing bone.

During this incorporation process, demineralization of the allograft material by infiltrating osteoclasts does not release any living cells into the recipient, therefore preventing a host-graft immune response. However, in the case of OsteoSpheres used for bone graft purposes, the possibility does exist that living cells (i.e. osteocytes, osteoblasts or osteoclasts) contained within the OsteoSpheres when released into the recipient's bone could initiate a host-graft response; therefore this may represent a significant barrier to the use of OsteoSpheres in non-autologous or non-tissue matched bone graft procedures. However, it is entirely possible that due to the slow rate and the small numbers of such “foreign” cells released at any given time from the mineralized matrix during incorporation of OsteoSpheres, a clinically significant host-graft immune response may not be mounted by the patient.

One approach to eliminate any possibility of a host-graft reaction is to produce OsteoSpheres using osteoblast and osteoclast cells that are not recognized as being foreign by the recipient's immune system. One means of achieving this goal is to develop human osteoblast and osteoclast pre-cursor cell lines that are incapable of generating a host-graft immune response due to the lack of human lymphocyte antigen (HLA) expression by these cells. As osteoblasts and osteoclasts are terminally differentiated cells, creation of such cell lines directly from these terminally differentiated cells may prove difficult due to the limited proliferative capacity of such cells.

Another approach to achieving the goal of osteoblast and osteoclast cell lines incapable of expressing HLA is to develop an osteogenic precursor cell line derived from embryonic or mesenchymal stem cells which are incapable of HLA expression. These cells can then in turn be differentiated into non-HLA expressing osteoblasts and osteoclasts for use in producing non-HLA expressing OsteoSpheres for bone grafting applications. Such osteogenic differentiation of stem cells has previously been achieved by exposing these cells to osteogenic differentiation factors such as BMP's. However to date, such simple exposure of stem cells to selected differentiation factors in tissue culture has resulted in varied levels of efficiency with regard to the numbers of cells that progress along the desired differentiation pathway.

A much more rapid and higher differentiation rates of stem cells may be achieved by mimicking the cellular milieu of the tissue to which differentiation is being driven. For example, human embryonic or mesenchymal stem cells that are grown on an irradiated feeder layer of murine bone marrow cells exhibit better osteogenic differentiation rates than those exposed to selected BMPs. However, exposure to such feeder layers poses significant contamination risks for stem cells destined for human clinical applications. Recent studies have shown that exposure of human embryonic stem cells to complex, soluble protein extracts of target tissues such as lung, drives differentiation towards the lung cell phenotype more efficiently. In addition, the presence of a membrane permeabilizing agent, polyethylene glycol, in the tissue culture medium significantly enhanced this response. These data suggest that optimal “driving” of the differentiation process towards a particular target tissue may be more efficient if the cells are cytoplasmically loaded with a mixture of complex proteins extracted from the cellular milieu to which they are being targeted.

Even if osteogenic differentiation can be enhanced to produce large numbers of human osteoblasts and osteoclasts, the issue surrounding the possibility of a host-graft response after implantation cannot be ignored. As mentioned above, one approach to overcoming the problem is the development of a stem cell line, either embryonic or adult, that has been genetically modified so as to not express the major proteins involved in “self-foreign” recognition at the cellular level, namely the human lymphocyte antigen family of cell surface proteins. Several approaches have previously been used for silencing of HLA expression in a variety of cell types. These include the introduction of RNA interference cassettes containing short hair-pin RNA (shRNA) sequences that target the gene encoding for the HLA Class 1 antigen. This approach has been demonstrated in a variety of cells including peripheral blood lymphocytes and human embryonic cells using lentivirus or adenovirus vectors to transfect the sequence into the recipient cells. The incorporation and transduction of these shRNA sequences by the transfected cell generates inhibitory RNA (RNAi) sequences that in turn block or “knock down” the HLA Class 1 expression in these cells without affecting the expression of other cellular proteins. Although this approach indicates that stable transfection of cells with specific inhibitory RNA sequences (RNAi) can be used as a means of silencing HLA expression, the use of lentivirus and adenoviral transfection vectors raises many safety concerns with regard to the potential neoplastic properties of these virally infected cells.

A related approach to silencing a particular gene is the production of “knock-out” cells that have been injected with partial DNA sequences corresponding to the gene of interest. This approach is used to generate “knock-out” or transgenic rodents by microinjection of partial or mutated DNA sequences directly into the nuclei of the cells of the blastocyst. These injected blastocysts are then implanted into females and allowed to gestate. During cell division, the partial DNA sequences recombine with the gene of interest, resulting in some cases in daughter cells from which this “mutated” gene has been excised from the genome. This process is random and does not always result in excision of the target gene. In those cases where excision does occur without inducing lethal disruption of the genome, the resulting surviving off-spring have cells with a normal genome absent the targeted gene and are referred to as “knock-out” animals for this particular gene.

A similar “knock-out” approach has been suggested for producing mesenchymal stem cells (MSC's) extracted from adult tissues such as bone marrow. These MSC's exhibit multi-potent properties and can be induced to undergo differentiation into a range of cell and tissue types, including bone and cartilage. Targeted disruption of gene expression in this fashion, such as the HLA family of genes, provides a possible means of generating an HLA negative MSC line that can be propagated indefinitely in culture and guided down the osteogenic cell differentiation pathway at will by exposing them to specific differentiation factors.

One of the persistent technical problems in producing either “knock-out” or “silenced” embryonic or adult stem cells has been the means of delivering the required genetic material to the cell. Use of plasmid constructs containing viral promoter sequences has raised many ethical issues concerning the subsequent use of such genetically modified cells in human clinical applications. In addition, chemical or irradiation-induced site mutagenesis raises the possibility of unintended collateral damage of the genome leading to unforeseen complications in these cells, such as the development of a neoplastic phenotype in vivo. As such, a means of delivering the required genetic material to the stem cell without the need to expose the cells to potentially harmful methods and/or reagents greatly enhances the possibility of using these genetically modified cells in human clinical applications.

Recently there has been described a highly efficient means of transferring a wide variety of macromolecules, such as dextrans, proteins, antibodies and plasmid DNA, directly into living cells without the need for exposure of the recipient cells to any chemical or viral vectors (U.S. Pat. No. 6,221,666). This technology, known as impact-mediated loading, is based on the concept that entry of large macromolecules into the cell cytoplasm that are normally impermeant to the cell plasma membrane, can be achieved by generating transient, survivable disruptions or “membrane wounds” of the plasma membrane. Such membrane wounds are a normal constitutive cellular response to mechanical loading of a variety of cell types in the body (i.e. cardiac myocytes, skeletal muscle myofibers, epithelial and endothelial cells) and provide a route through which a macromolecule can directly enter the cell cytoplasm from the extra-cellular fluid by diffusion. However, such constitutive membrane wounds are normally small and provide limited communication between the extra-cellular environment and the cell cytoplasm.

By developing the impact loading technique and apparatus for producing reproducible membrane wounds of relatively uniform size in a controlled fashion, gaining direct access to the cell cytoplasm became routinely possible. In addition, we also demonstrated that the efficiency of this transfer process (in terms of overall cell survival, the amount of macromolecule transferred and the size of the macromolecule that can be transferred) is significantly improved if the impact-mediated loading process is carried out under hyper-gravity conditions. Furthermore, unlike a variety of other techniques used for direct transfer of protein or genetic material into the living cell cytoplasm, this technique is applicable to all cell types including primary cells of both human and animal origin, producing significantly higher yields of loaded cells in a shorter period of time than competing technologies.

For example, this technique has been used to load a variety of macromolecules including large proteins, IgG antibodies and inert dextrans (up to 2×10⁶ daltons in size) directly into primary human cells. The technique has also been used to achieve transfection of primary human cells with plasmid DNA constructs normally resistant to such transfer protocols. The impact-mediated loading technique allows large numbers of cells (>10,000 cells) to be simultaneously loaded in a matter of seconds, with cell survival rates approaching 95%-100% of the starting cell population depending on the loading conditions; the smaller the molecule being loaded the higher overall loading/transfection efficiency that can be achieved. As RNAi sequences and partial DNA sequences are relatively small in terms of their molecular size, these compounds are ideally suited for delivery using the impact-mediated loading technology. As such, this technique is ideally suited for the production of genetically modified human embryonic stem cells without the need for the use of chemical or viral transfection agents that may modify the cells in unwanted or unanticipated ways.

Described herein is a method for producing a human embryonic stem cell line or an adult stem cell line of mesenchymal origin in which at a minimum HLA Class I protein expression has been silenced: (1) by the stable expression of an inhibitory RNA sequence for the human HLA Class I gene (HLA1-RNAi) contained in a shRNA cassette transfected into these cells; or (2) as a consequence of HLA gene “knock-out” by delivery of a partial DNA sequence of the HLA gene of interest Inhibitory RNAi sequences for HLA Class I, shRNA transfection cassettes and the DNA sequence for the HLA Class I gene have previously been described in the literature. Such RNAi sequences, partial DNA sequences and shRNA cassettes are now available from a variety of commercial sources in purified form. These cells may also be transfected with similar shRNA cassettes containing the inhibitory RNA sequences for additional human HLA genes or other “self-foreign” recognition genes such as beta-microglobulin. Transfection of embryonic or adult stem cells may be achieved by loading the cells with purified shRNA cassettes encoding for these RNAi sequences to HLA genes using the impacted mediated loading technology known as The G-Loader. The G-loader device itself is approximately 25 cm tall and 10 cm in diameter (see FIG. 8). A 35 mm tissue culture plate containing a monolayer of adherent cells is placed in the base of the G-Loader after being incubated with loading medium containing the macromolecule of interest. Prior to placing the cell sample into the G-Loader, a particle cartridge is inserted into the device consisting of a circular cartridge that supports a rupturable membrane on which is located a layer of 10 micron particles arranged in a specific pattern (see panel insert). In addition, the device has been charged with air to the required pressure and the on-board g-load accelerometer trigger has been set to the required g-value using the G-Loader Charging Station (for bench top operation there is a manual trigger button located on the side wall of the device). When the G-Loader is activated, either manually or by reaching a set g-load a metered volume of pressurized air is directed into the particle cartridge, the membrane ruptures and disperses the particles into the air stream. The particles then impact the cell layer inducing membrane wounding in a high controllable and reproducible fashion.

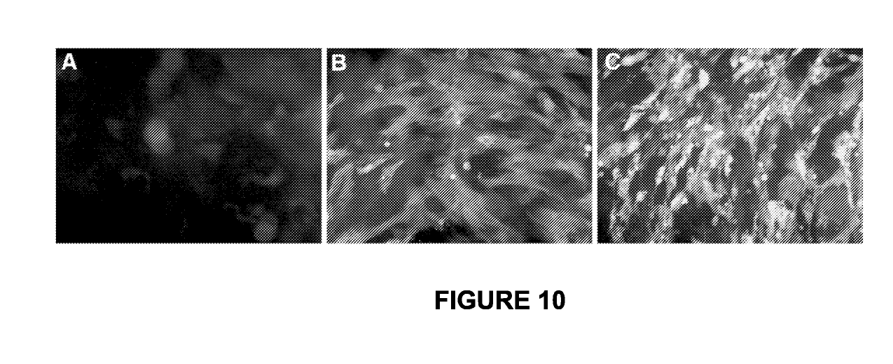

FIG. 10 shows enhanced impact-mediated loading under hypergravity conditions. Specifically, FIG. 10 shows fluorescent micrographs of Swiss 3T3 cell monolayers cytoplasmically loaded with Fluor™ 488 goat-anti-mouse IgG employing impact-mediated loading at 1×g on the bench-top (Panel B) and at 200×g in a bench top centrifuge (Panel C). Control cells (Panel A) were exposed to IgG for the same period of time but were not impact loaded. Panels A, B and C were taken at the same photographic conditions. Note the larger amounts of IgG present in the cytoplasm of cells loaded at 200×g as compared to those loaded at 1×g and that IgG is excluded from the nuclear regions of the loaded cells.

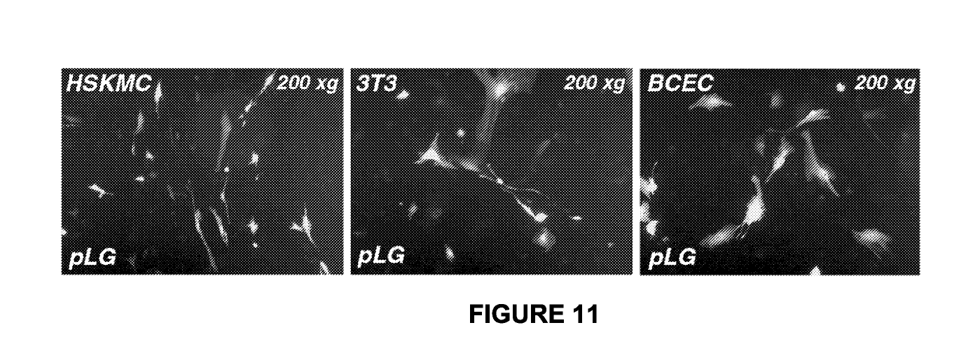

FIG. 11 shows fluorescent micrographs of primary human skeletal myoblasts (HSKMC), Swiss 3T3 cells (3T3) and primary bovine capillary endothelial cells (BCEC) 24 hr after impact-mediated loading of a plasmid construct encoding for a green fluorescent protein, Lantern Green™ (pLG).

Alternatively, DNA sequences can be directly transferred to the cell nuclei using standard microinjection techniques. Transfection efficiency using the impact-mediated loading technique may be optimized in these cell types by mechanistically selecting the optimal impact velocity, size of the impact particle and the optimal g-load at which to perform the transfer. The transfer process may also be enhanced by pre-treatment of the cell surface prior to loading with naturally-derived hyaluronidase enzyme and/or a low molecular weight surfactant such as PF-68 to increase access of the purified genetic material to the cell membrane surface. In addition, co-loading of low molecular weight surfactants, such as PF-68, appear to enhance transfection efficiency by a mechanism postulated to involve encapsulation of the genetic material in a protective layer of surfactant thereby inhibiting intracellular degradation prior to expression. Such an approach may also increase the degree of random transfer of partial DNA sequences to the cell nuclei from the cytoplasm of loaded cells.

After transfection with either RNAi sequences or partial DNA sequences for HLA genes, cells are returned to their standard culture conditions for a period of three days. At this time, the loaded cell population are cloned by limiting dilution and the resulting clones are again expanded in culture until such time that enough cells have been generated in order to test for the expression of specific RNAi, RNA or DNA sequences in extracts of a portion of the cloned cell population. Depending on the genetic material delivered to the original cells, these clones are also tested for: (1) the presence of the HLA RNAi sequence, and (2) the absence of RNA encoding the HLA gene; or (3) the absence of the DNA sequence encoding the HLA gene. Those clones showing evidence of (1) HLA RNAi sequence expression and a lack of HLA gene RNA expression or (2) the lack of HLA gene DNA sequences, are further expanded in tissue culture while cryogenically preserving portions of the cloned cell population.

Following a suitable period of time to allow expansion of the cloned cells, a sample of cells from the surviving clones is exposed to tissue culture medium containing osteoblastic differentiation agents, such as ascorbic acid, beta-phosphoglycerol, hydrocortisone-21-hemisuccinate, BMP-2, BMP-4 and/or BMP-7, for a period of seven days and then tested for the production osteoblastic cell markers such as alkaline phosphatase and osteocalcin. In addition, a sample of these cloned cells is also exposed to tissue culture medium containing osteoclastic differentiation agents (i.e. RANKL, M-CSF) and then tested for the production of osteoclastic cell markers such as fluoride-sensitive tartrate-resistant acidic phosphatase (TRAP).

As an alternative means of driving osteogenic cell differentiation by simple exposure of the cloned stem cells to various selected compounds dissolved in their tissue culture medium, cloned stem cells are also cytoplasmically loaded with a complex soluble protein extract of primary human osteoblast or osteoclast cultures using the impact mediated loading technology. The cells are then cultured for a further seven days in culture and tested for osteoblastic and osteoclastic markers as detailed above.

Depending on the genetic material delivered to the original cells from which these clones were derived, the differentiated cells are also tested for the absence of either DNA encoding the HLA gene or mRNA encoding HLA proteins or for the presence of RNAi sequences directed against the HLA genes. Criteria for selecting cloned cells for further expansion include the following: (1) positive for the HLA RNA sequence and negative for HLA mRNA or (2) negative for the DNA HLA gene sequence; and (3) the ability to differentiate into osteoblasts or osteoclasts depending on the differentiation stimulus provided.

Those clones that are capable of differentiating into osteoblasts and/or osteoclasts are further expanded in order to develop a cell line in which HLA expression has been silenced by stable transfection of RNAi sequences against the HLA gene(s) or by HLA gene knockout. These cells are then differentiated into osteoblasts and osteoclasts and combined using the methods of the preceeding examples to produce OsteoSpheres for clinical use in bone graft procedures. Cell material is extracted from fully mature, mineralized OsteoSpheres. Depending on the genetic material delivered to these cells, this material is tested for the expression of HLA RNAi sequences and the absence of HLA mRNA or the absence of the HLA DNA gene sequence. In addition, all samples are tested by protein immuno-blotting for the absence of actual HLA protein in order to determine that the constituent cells of the OsteoSpheres are indeed negative for HLA expression at both the genetic and protein levels.

What is claimed is:

1. A method for producing a human osteogenic cell line in which HLA Class I expression is silenced, the method comprising:
providing adult mesenchymal stem cells or embryonic mesenchymal stem cells;
introducing into said adult mesenchymal stem cells or said embryonic mesenchymal stem cells a short hairpin RNA (shRNA) cassette capable of directing expression of an inhibitory RNA specific for the HLA Class I gene; and
identifying and propagating individual cells in which the HLA Class I RNA gene is silenced.

2. The method of claim 1 wherein said shRNA cassette is introduced into said adult mesenchymal stem cells or said embryonic mesenchymal stem cells using impact-mediated loading.

3. A human osteogenic line produced by a method comprising:
providing adult mesenchymal stem cells or embryonic mesenchymal stem cells;
introducing into said adult mesenchymal stem cells or said embryonic mesenchymal stem cells a short hairpin RNA (shRNA) cassette capable of directing expression of an inhibitory RNA specific for the HLA Class I gene; and
identifying and propagating individual cells in which the HLA Class I RNA gene is silenced.

4. The human osteogenic line of claim 3 wherein said shRNA cassette is introduced into said adult mesenchymal stem cells or said embryonic mesenchymal stem cells using impact-mediated loading.

5. A method for producing a human osteogenic cell line in which HLA Class I expression is silenced, the method comprising:
providing adult mesenchymal stem cells or embryonic mesenchymal stem cells;
introducing into said adult mesenchymal stem cells or said embryonic mesenchymal stem cells a DNA specific for the HLA Class I gene wherein said DNA disrupts said HLA Class I gene as a result of site specific recombination; and
identifying and propagating individual cells in which the HLA Class I gene is silenced.

6. The method of claim 5 wherein said DNA specific for the HLA Class I gene is introduced into said adult mesenchymal stem cells or said embryonic mesenchymal stem cells using impact-mediated loading.

7. A human osteogenic line produced by a method comprising:
providing adult mesenchymal stem cells or embryonic mesenchymal stem cells;
introducing into said adult mesenchymal stem cells or said embryonic mesenchymal stem cells a DNA specific for the HLA Class I gene wherein said DNA disrupts said HLA Class I gene as a result of site specific recombination; and
identifying and propagating individual cells in which the HLA Class I gene is silenced.

8. The human osteogenic line of claim 7 wherein said DNA specific for the HLA Class I gene is introduced into said adult mesenchymal stem cells or said embryonic mesenchymal stem cells using impact-mediated loading.

9. A method for producing mineralized three-dimensional bone constructs, the method comprising:
providing a human osteogenic cell line in which HLA Class I expression is silenced;
differentiating cells in said human osteogenic cell line into osteoblasts and osteoclast precursors;
introducing said osteoclast precursors and said osteoblasts into a cylindrical culture vessel that rotates about a central horizontal axis, said cylindrical culture vessel comprising a matrix-free culture medium;
culturing said osteoblasts and said osteoclast precursors in said cylindrical culture vessel during horizontal rotation at a rate effective to create low shear conditions and to promote the formation of aggregates comprising said osteoclast precursors and said osteoblasts;
further culturing said aggregates in said cylindrical culture vessel during horizontal rotation at a rate effective to create low shear conditions, whereby said aggregates grow in size and said osteoclast precursors differentiate into osteoclasts;
introducing a matrix-free mineralization culture medium into said cylindrical culture vessel and culturing said aggregates during horizontal rotation at a rate effective to create low shear conditions, wherein mineralized three-dimensional bone constructs are formed.

10. The method of claim 9 wherein providing said human osteogenic cell line comprises:
providing adult mesenchymal stem cells or embryonic mesenchymal stem cells;
introducing into said adult mesenchymal stem cells or said embryonic mesenchymal stem cells a short hairpin RNA (shRNA) cassette capable of directing expression of an inhibitory RNA specific for the HLA Class I gene; and
identifying and propagating individual cells in which the HLA Class I RNA gene is silenced.

11. The method of claim 10 wherein said shRNA cassette is introduced into said adult mesenchymal stem cells or said embryonic mesenchymal stem cells using impact-mediated loading.

12. The method of claim 9 wherein providing said human osteogenic cell line comprises:
providing adult mesenchymal stem cells or embryonic mesenchymal stem cells;
introducing into said adult mesenchymal stem cells or said embryonic mesenchymal stem cells a short hairpin RNA (shRNA) cassette capable of directing expression of an inhibitory RNA specific for the HLA Class I gene; and
identifying and propagating individual cells in which the HLA Class I RNA gene is silenced.

13. The method of claim 12 wherein said shRNA cassette is introduced into said adult mesenchymal stem cells or said embryonic mesenchymal stem cells using impact-mediated loading.

14. A mineralized three-dimensional bone construct produced by a method comprising:
providing a human osteogenic cell line in which HLA Class I expression is silenced;
differentiating cells in said human osteogenic cell line into osteoblasts and osteoclast precursors;
introducing said osteoclast precursors and said osteoblasts into a cylindrical culture vessel that rotates about a central horizontal axis, said cylindrical culture vessel comprising a matrix-free culture medium;
culturing said osteoblasts and said osteoclast precursors in said cylindrical culture vessel during horizontal rotation at a rate effective to create low shear conditions and to promote the formation of aggregates comprising said osteoclast precursors and said osteoblasts;
further culturing said aggregates in said cylindrical culture vessel during horizontal rotation at a rate effective to create low shear conditions, whereby said aggregates grow in size and said osteoclast precursors differentiate into osteoclasts;
introducing a matrix-free mineralization culture medium into said cylindrical culture vessel and culturing said aggregates during horizontal rotation at a rate effective to create low shear conditions, wherein mineralized three-dimensional bone constructs are formed.

15. The mineralized three-dimensional bone construct of claim 14, wherein providing said human osteogenic cell line comprises:
providing adult mesenchymal stem cells or embryonic mesenchymal stem cells;
introducing into said adult mesenchymal stem cells or said embryonic mesenchymal stem cells a short hairpin RNA (shRNA) cassette capable of directing expression of an inhibitory RNA specific for the HLA Class I gene; and
identifying and propagating individual cells in which the HLA Class I RNA gene is silenced.

16. The method of claim 15 wherein said shRNA cassette is introduced into said adult mesenchymal stem cells or said embryonic mesenchymal stem cells using impact-mediated loading.
